(19) United States
(12) Patent Application Publication
(10) Pub. No.: US 2007/0207227 A1
(43) Pub. Date: Sep. 6, 2007

(54) APPETITE SUPPRESSANT COMPOSITIONS

(75) Inventors: Salomon Leendert ABRAHAMSE, Vlaardingen (NL); Kevin John POVEY, Purfleet (GB); Daryl David REES, Cambs (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., D/B/A UNILEVER, A CORPORATION OF NEW YORK, Englewood Cliffs, NJ (US)

(21) Appl. No.: 11/458,698

(22) Filed: Sep. 12, 2006

(30) Foreign Application Priority Data
Feb. 23, 2006 (EP) 06110333.9
Jun. 13, 2006 (EP) 06115404.3

(51) Int. Cl. A61K 36/27; A61K 31/704

(52) U.S. Cl. 424/769; 514/26

(57) ABSTRACT

An inherently nourishing edible appetite suppressant product comprising a steroidal glycoside, preferably obtainable from plants of the Asclepiadaceae family, the product being in the unit serving, having a weight of from 20 to 600 grams, and having the energy density of from 1.6 to 23 kilojoules per gram of product. A dietary regimen is also disclosed.

### TECHNICAL FIELD

The present invention relates generally to appetite suppressant compositions. More in particular, it relates to appetite suppressant compositions comprising steroidal glycosides, preferably from plants of the Asclepiadaceae family.

### BACKGROUND OF THE INVENTION

In the Western world, but also in other countries where a so-called Western diet is adopted, the incidence of being overweight and obese has drastically increased over the last decades. “Overweight” and “obesity” are both labels for ranges of body weight that are greater than what is generally considered healthy for a given height. The terms also identify ranges of weight that have been shown to increase the likelihood of certain diseases and other health problems. For adults, overweight and obesity ranges are determined by using weight and height to calculate a number called the “body mass index” (BMI). BMI is used because, for most people, it correlates with their amount of body fat. An adult who has a BMI between 25 and 29.9 is considered overweight. An adult who has a BMI of 30 or higher is considered obese.

Since obesity and being overweight are generally known to be associated with a variety of diseases such as heart disease, type 2 diabetes, hypertension and artherosclerosis, this increase is a major health concern for the medical world and for individuals alike. Furthermore, people having a pronounced overweight state may consider themselves as unattractive, which leads to a clearly reduced feeling of well-being.

This has led to an increasing interest by consumers in their health and has created a demand for products that help to reduce or control daily caloric intake and/or control body weight and/or bodily appearance.

Several solutions have been proposed to help individuals to control their weight. Among these solutions is the use of drugs e.g. to suppress the activity of enzymes in the digestive system. However, the use of drugs is often not preferred unless strictly required for medical purposes.

Another proposed solution is to prescribe the individuals a specific diet, for example, a diet with a restricted caloric intake per day. A problem with these diets is that often they do not provide a healthy nutritional balance and/or they are difficult to accommodate in modern lifestyles.

Meal replacement products have also been proposed as part of a healthy diet in order to control or reduce body weight. For example, U.S. Pat. No. 5,688,547 discloses a nutritional meal replacement composition comprising dietary fibre, protein and a cellulose gum and gel. These meal replacement products are generally products that are intended to be consumed as a single-serving food product, such as a bar, drink etc to replace one or two meals per day. The meal replacement products are designed such that on the one hand they provide a restricted caloric intake, but on the other hand they provide a healthy balance of nutritional ingredients and are convenient to incorporate into an individual's daily diet. Commercial meal replacement products include, for instance, the Slim-Fast® brand (http://www.slim-fast.com).

However, a general problem with products intended for use in a weight loss or weight maintenance plan, e.g. meal replacement products or low-calorie snacks, is that food intake is not sufficiently reduced and/or appetite is not sufficiently suppressed after consumption and/or the feeling of satiety obtained may not be as great as desired. These factors may render it difficult for the individual to adhere to the plan or it may make it and/or the products used therein less appealing to consumers.

Another approach is to use appetite suppressant compositions that actually diminish the appetite, European Patent EP-A 994 655 discloses appetite suppressant compositions comprising an emulsion containing palm oil, oat oil and water.

Extracts from plants of the Asclepiadaceae family, particularly the Hoodia genus (formerly the Hoodia and Trichocaulon genera) have also been shown to have an appetite suppressant activity. U.S. Pat. No. 6,376,657 discloses that these plants contain steroidal glycosides having the formula 1:

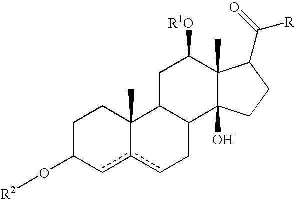

wherein

R=alkyl;

R¹═H, alkyl, tiglyol, benzoyl or any other organic ester group;

R²═H or one or more 6-deoxy carbohydrates, or one or more 2,6-dideoxy carbohydrates, or glucose radical or combinations thereof; and wherein the broken lines indicate the optional presence of a further bond between carbon atoms C4 and C5 or between carbon atoms C5 and C6.

U.S. Pat. No. 6,376,657 also discloses processes to extract steroidal glycosides having the formula 1 from plants of the Asclepiadaceae family. Also disclosed are synthetic methods of obtaining steroidal glycosides of Formula 1 and analogs thereof.

WO2005/116049 (Unilever) discloses that steroidal glycosides can be extracted or separated from undesirable components present in plant material of the Asclepiadaceae (Hoodia) family by means of liquid or supercritical carbon dioxide.

US 2005/0202103 (Rajendran et al.) discloses steroid glycosides obtainable from Caralluma, another genus of plants in Asclepiadaceae family. U.S. Pat. No. 7,008,648 (Corley et al.) discloses steroidal glycosides obtainable from Stapelia and Orbea plants. WO 2005/099737 discloses additional steroid glycosides obtainable from Asclepias plant.

US 2006/0083795 discloses meal replacement products containing some plant extracts, including extracts from Hoodia.

Incorporating an appetite suppressant into the diet creates new challenges for the product developer to provide the correct conditions to achieve weight loss through appetite suppression, yet also maintain an adequate level of nutrition, since living organisms do need to continue eating in order to live and to be well. For example, a product developer must avoid consumers cutting back too much on nutrition intake, as the result of appetite reduction. A healthy balance needs to be attained between suppressing appetite, yet maintaining the healthy intake of energy sources and also balanced nutrition with an adequate level of fat, protein and carbohydrate.

Accordingly, there is still a need for appetite suppressant products, which are inherently nourishing providing at least a minimum of energy intake, yet also suppress appetite and limit overeating. In particular, there is a need for such appetite suppressant products comprising steroidal glycosides.

We have now surprisingly found that these and other objects of the invention may be achieved by an edible appetite suppressant product according to the invention comprising a steroidal glycoside, the product being in unit serving form, having a weight of from 20 to 600 grams, and having the energy density of from 1.6 to 23 kilojoules per gram of product.

### SUMMARY OF THE INVENTION

In a first aspect, the present invention relates to an edible appetite suppressant product in unit serving form comprising from 5 to 5000 milligrams of a steroidal glycoside, the product having a weight of from 20 to 1000 grams, and having the energy density of from 1.6 to 23 kilojoules per gram of product.

The invention is based at least in part on the discovery that the inventive product ensures that the appetite suppressant cannot be ingested without the consumer getting a minimum level of energy intake, preferably in the form of a healthy balance of fats, carbohydrates and proteins. The inventive product provides healthy weight loss, yet avoids too rapid weight loss or insufficient energy intake by anorexics or extreme dieters.

In a second aspect, the invention relates a method of using such compositions for suppressing appetite and/or controlling obesity.

In a third aspect, the invention relates to a dietary regimen which balances the appetite suppression (and/or weight loss)on one hand and provides at least a minimum of energy on the other hand, which regimen is described by the Energy Balance Equation (discussed in greater detail below).

### BRIEF DESCRIPTION OF THE DRAWINGS

FIG. 1 shows the daily food intake of rats that received various amounts of Hoodia gordonii, as described in detail in Example 6.

FIGS. 2 and 3 illustrate the effects of simultaneous consumption of a nourishing amount of energy and steroidal glycosides on energy intake in humans, as described in Example 7.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration or amount, any particular upper concentration can be associated with any particular lower concentration or amount.

For the avoidance of doubt the word “comprising” is intended to mean “including” but not necessarily “consisting of⇄ or “composed of.” In other words, the listed steps or options need not be exhaustive.

“Unit serving” as used herein means separately packaged individual servings of food or drink.

“Energy Density” as used herein means the number of kilojoules (kJ) per gram (g) of the product.

### Steroidal Glycoside

The inventive edible appetite suppressant compositions comprise a steroidal glycoside. “Steroidal glycoside” as used herein means a steroid (four fused rings), further comprising at least one side group substitution which is a glycoside (a molecule in which a sugar group is bonded through its anomeric carbon to another group via an O-glycosidic bond), preferably a deoxy or di-deoxy glycoside.

The steroidal glycoside may be synthetically produced or it is obtainable from plants. Synthetic methods are disclosed in the U.S. Pat. No. 6,376,657, incorporated by reference herein. Preferably, the steroidal glycoside is obtainable from plants of the Asclepiadaceae family. Most preferably, the steroidal glycoside is delivered to the inventive compositions in the form of an extract from the plants.

Suitable plants include but are not limited to Hoodia, Caralluma, Orbea, Stapelia, Lavrania genera of plants, and mixtures thereof.

Most preferably, the steroidal glycoside is extracted from plants of the genus Trichocaulon or of the genus Hoodia, as described in U.S. Pat. No. 6,376,657, incorporated by reference herein. Other methods of preparing an extract are also acceptable.

More preferably, the plant extract is selected from the group consisting of appetite suppressant Trichocaulon piliferum extracts, appetite suppressant Trichocaulon officinale extracts, appetite suppressant Hoodia currorii extracts, appetite suppressant Hoodia gordonii extracts, appetite suppressant Hoodia lugardii extracts and mixtures thereof.

Suitable steroidal glycoside compounds include but are not limited to the general structural formulae (1)-(5):

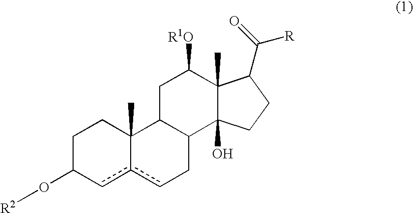

wherein

R=alkyl;

R¹═H, alkyl, tiglyol, benzoyl or any other organic ester group;

R²═H or one or more 6-deoxy carbohydrates, or one or more 2,6-dideoxy carbohydrates, or glucose radical, or combinations thereof; and wherein the broken lines indicate the optional presence of a further bond between carbon atoms C4 and C5 or between carbon atoms C5 and C6.

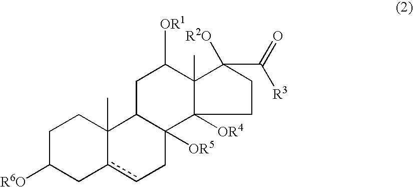

wherein R¹ is hydrogen or a C₁-C₁₈ radical;

R² is hydrogen or a C₁-C₁₈ radical;

R³ is a C₁-C₁₈ radical;

R⁴ is hydrogen or a C₁-C₁₈ radical;

R⁵ is hydrogen or a C₁-C₁₈ radical;

R⁶ is hydrogen, a C₁-C₁₈ radical, or a saccharide radical; and,

the dotted line represents an optional double bond.

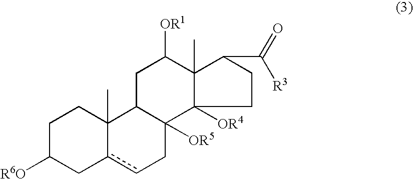

wherein R¹ is hydrogen or a C₁-C₁₈ radical;

R³ is a C₁-C₁₈ radical;

R⁴ is hydrogen or a C₁-C₁₈ radical;

R⁵ is hydrogen or a C₁-C₁₈ radical;

R⁶ is hydrogen, a C₁-C₁₈ radical, or a saccharide radical; and,

the dotted line represents an optional double bond.

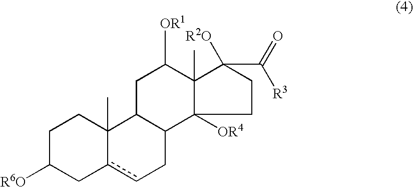

wherein R¹ is hydrogen or a C₁-C₁₈ radical;

R² is hydrogen or a C₁-C₁₈ radical;

R³ is a C₁-C₁₈ radical;

R⁴ is hydrogen or a C₁-C₁₈ radical,

R⁶ is hydrogen, a C₁-C₁₈ radical, or a saccharide radical; and,

the dotted line represents an optional double bond.

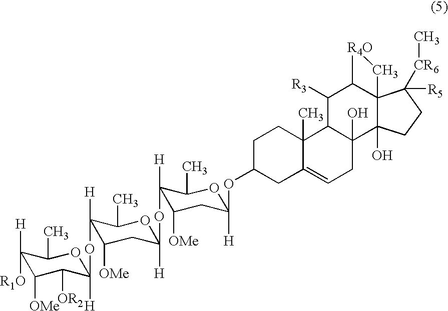

wherein R₁-R₆ have the following meaning:

| Compound | R₁ | R₂ | R₃ | R₄ | R₅ | R₆ |
|---|---|---|---|---|---|---|
| Stavaroside A | H | H | H | Ang | OH | H, O—Bz |
| Stavaroside B | H | H | H | Ang | OH | H, O—Tig |
| Stavaroside C | H | H | O—Ac | Bz | H | ═O |
| Stavaroside D | H | H | O—Ac | Tig | H | ═O |
| Stavaroside E | H | H | H | Bz | OH | H, OH |
| Stavaroside F | H | H | O—Ac | Ac | H | ═O |
| Stavaroside G | H | H | H | Ac | OH | H, O—Ac |
| Stavaroside H | H | H | OH | H | H | ═O |
| Stavaroside I | β-D- | H | O-Ang^{a} | Bz^{a} | H | ═O |
| | glc | | | | | |
| Stavaroside G | β-D- | H | O—Ac | Bz | H | ═O |
| | glc | | | | | |
| Stavaroside H | β-D- | H | O—Ac | Tig | H | ═O |
| | glc | | | | | |

^{a}Interchangeable
Ac = Acetate
Ang = Angelate
Bz = Benzoate
Tig = Tiglate
β-D-glc = β-D-glucopyranosyl

Particularly preferred steroidal glycosides are analogs of Compound of Formula I, whether synthetically produced or extracted from the plants, including Compounds of Formula (6) through Formula (12), and mixtures thereof, since these are obtainable from the preferred Hoodia plants.

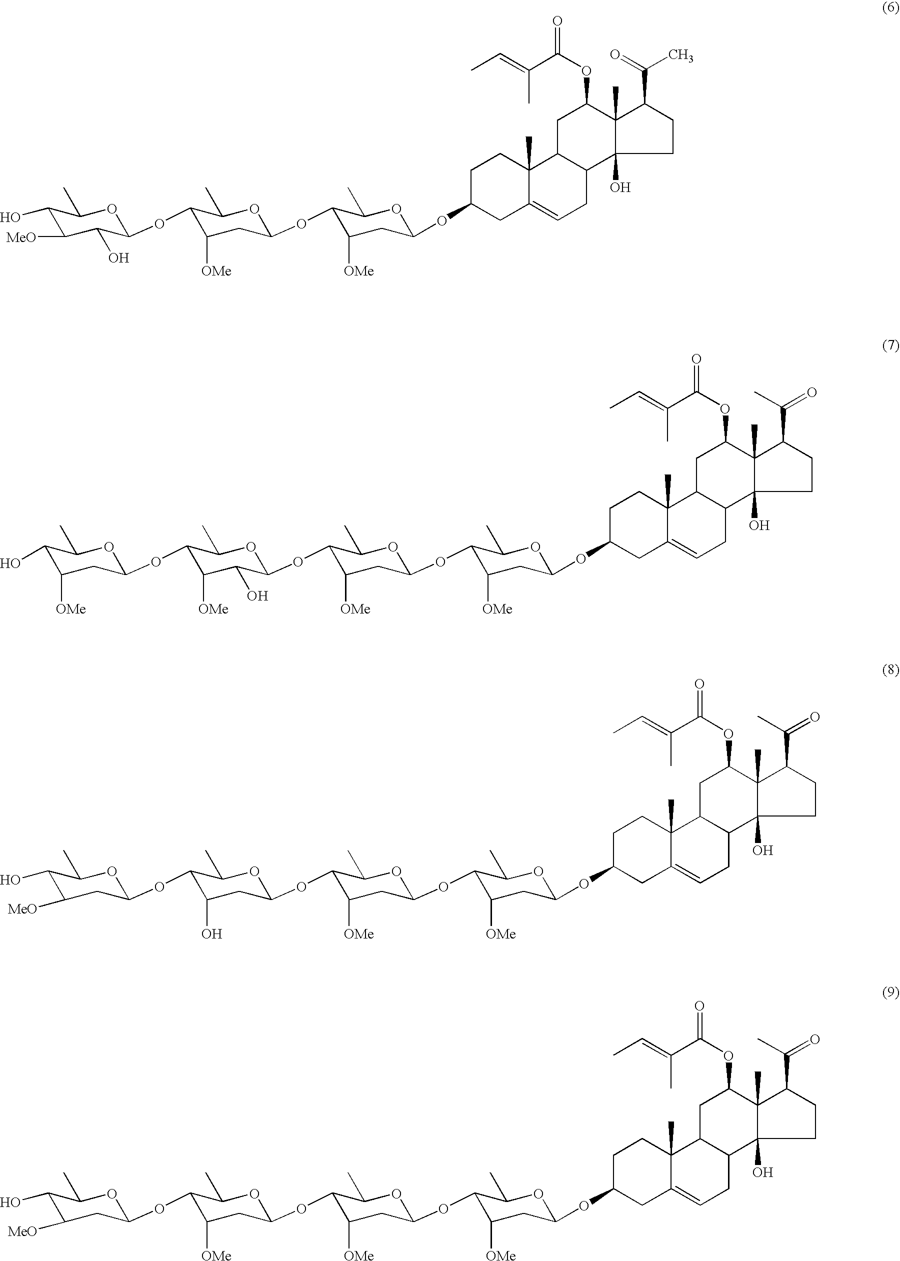

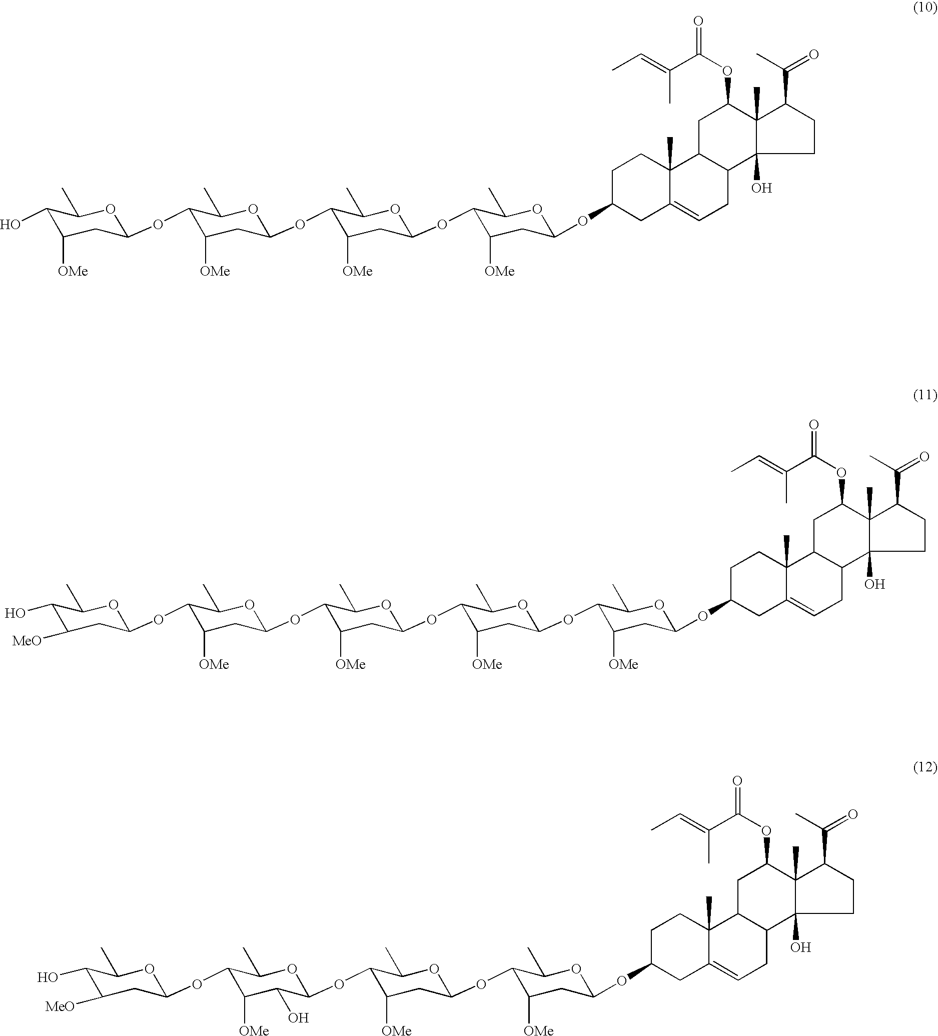

Other steroidal glycosides not specifically mentioned herein may be included in the inventive product. It will be understood that the invention also encompasses isomers, derivatives, salts, esters and analogs of the steroidal glycosides (preferably, biologically active) and mixtures thereof.

The product according to the invention comprises from 5 to 5000 milligrams (hereinafter “mg”) of a steroidal glycoside, preferably from 10 to 3000 mg, more preferably from 20 to 2500 mg, and most preferably from 50 to 2000 mg in order to provide the optimum and consistent appetite suppressant response among the population.

When an extract is used, the extract comprises at least 10%, by weight of the extract, of steroidal glycosides, preferably at least 20%, most preferably at least 60%, and optimally at least 70%. U.S. Pat. No. 6,376,657, incorporated by reference herein, describes the preparation of a suitable extract comprising steroidal glycosides from the genus Hoodia, said steroidal glycoside having appetite suppressant activity. The solvents specifically mentioned to perform the extraction are one or more of methylene chloride (dichloromethane), water, methanol, hexane, ethyl acetate or mixtures thereof. An alternative method to obtain an extract is disclosed by separating plant sap from the plant solid material. “Extract” as used herein includes liquid, solid or spray-dried forms of extracts, sap, which also may be purified, partially purified, concentrated and/or fractionated. Other methods of extracting a steroidal glycoside from plants are also suitable.

Besides the extract, the steroidal glycoside may be incorporated in the form of otherwise concentrated preparation, e.g. a dried plant or otherwise concentrated plant preparation, which preferably contains the same steroidal glycoside amounts as described above with respect to the extract.

### Energy Density

The product of the invention has the energy density of from 1.6 to 23 kilojoules (“kJ”) of energy per gram (“g”) of the product (the energy density also sometimes referred to herein as “kJ/g”), preferably from 2 to 20 kJ/g, most preferably from 1.6 to 10 kJ/g for unit serving drinks and from 10 to 20 kJ/g for unit serving bars, in order to deliver optimum weight control and nutritional benefits. The energy in the inventive product is delivered through carbohydrates, fats, proteins and mixtures thereof, preferably through an optimised healthy balance of the carbohydrates, fats, and proteins, as described below.

Suitable carbohydrates include but are not limited to potato, pasta, wheat, corn, soy fiber, fruit fiber (e.g. apple and orange),sucrose, fructose, dextrose, lactose, maltodextrins, honey, corn syrup, oligofructose, starches (e.g potato starch, corn starch, rice starch), modified starches, fruit juice, concentrated fruit juice, flours (e.g wheat, corn and rice), gums (e.g. xanthan gum, guar gum, gum arabic, locust bean gum, celluloses (e.g sodium carboxy methyl cellulose, microcrystalline cellulose, powdered cellulose), carageenan, potassium carageenan, alginates (e.g sodium and potassium alginates), gelatin, pectin and mixtures thereof.

It should be noted that some or all of the sugar may be replaced by artificial sweeteners, or artificial sweeteners may be present in addition to sugars. Artificial sweeteners include but are not limited to aspartame, cyclamates (e.g. Sodium cyclamate), acesulfame K, sucralose, saccharin, invert sugar, maltose sugar, sugar alcohols (e.g maltitol, sorbitol)and mixtures thereof.

Suitable fats include but are not limited to saturated and unsaturated fats and oils, for instance sunflower oil, high oleic sunflower oil, canola, cottonseed oil, corn/maize oil, rapeseed oil, olive oil, soybean oil, palm oil, palm kernel oil, coconut, fish oil, linseed oil, peanut/groundnut oil, safflower oil, sesame oil, butter, lard, cocoa butter, mono and diglycerides and mixtures thereof.

The sources of oils and fats can also be hardened (e.g. by hydrogenation) or fractionated (e.g. via solvents) and these can be mixed with other oils or fats.

In order to optimise the health value of the inventive products, at least some of the fat, generally from 10 to 80%, preferably from 30 to 50%, by weight of the total fat, is present as unsaturated or polyunsaturated oil, in particular oils which contain linoleic (e.g sunflower, soybean, corn, Linola™ or rapeseed) or linolenic acid (e.g linseed) and mixtures thereof. Ideally the product should deliver at least 1 g per day of linoleic and/or linolenic acid, preferably in the form of a triglyceride from an unsaturated fat source.

To minimise the potential harmful effects, the inventive products are substantially free of trans fat, i.e. contain less then 0.5% of trans fat, preferably less then 0.1%, most preferably less than 0.05% and optimally 0% trans fat, by weight of the product.

Suitable proteins include but are not limited to milk, skimmed milk, fat free milk, condensed milk, fermented milk, cream, whey, yoghurt, cheese, egg, buttermilk, milk powder, buttermilk powder, cream powder, whey powder, yoghurt powder, cheese powder, egg powder, calcium and sodium caseinates, lactose free dairy proteins, soy proteins, isolated soy proteins, vegetable proteins, meat and fish derived proteins, gelatin, albumin powder, and mixtures thereof.

The general and preferred ranges for the relative amounts of energy from fat and protein in the inventive product is as follows:

| Ranges | % energy from fat | % energy from protein |
|---|---|---|
| General | 0.5–55 | 3–50 |
| Preferred | 2–45 | 5–45 |
| Most Preferred | 5–40 | 10–40 |
| Optimal | 10–30 | 10–35 |

Typically, carbohydrates deliver the balance of the energy requirement. Generally, this means according to the present invention, that carbohydrates would contribute between 10 and 75% energy in the product. At the preferred, most preferred and optimal ranges, the best nutritional benefit may be delivered at an optimum cost.

### Method of Using

The inventive product is used for suppressing appetite and/or controlling obesity in humans, while at the same time providing at least a minimum nutrition and a balanced intake of proteins, fats, and carbohydrates.

Generally, at least one inventive product should be ingested per day, typically from one to five per day (per 24 hours), until reaching the desired weight, and then continuing with this regime to maintain the desired weight. Most preferably, the invention is used from 1 to 3 times per day for optimum effect.

The invention also includes a dietary regimen which balances the appetite suppression (and/or weight loss) on one hand and provides at least a minimum of energy on the other hand,

which regimen is described by the Energy Balance Equation as follows:

Eᵣ=(Eₘ*[Hₑ]*s)/(H₅₀]+[Hₑ]*s)+Eₛ*s

where,

Eᵣ is the reduction in daily energy intake when consuming a certain number of unit servings (in kJ)

Eₘ is the maximum reduction in daily energy intake (in kJ) when consuming an infinite number of unit servings consisting of active steroidal glycosides and water (i.e. no energy).

[Hₑ] is the amount of active steroidal glycosides in the unit serving (in mg)

H₅₀ is the amount of active steroidal glycosides (in mg) that gives a 50% reduction in energy intake when consumed in a unit serving form without energy

Eₛ is the amount of energy in the unit serving (in kJ)

s is the number of unit servings consumed.

H₅₀ depends on the absorption of the active steroidal glycosides from the unit serving and will depend on the composition of the unit serving. Hₑ and Eₛ can be varied to give an optimal reduction in energy intake yet also delivering a nourishing amount of energy.

The regimen according to the invention attains Eᵣ energy intake reductions of 500 to 5000 kJ per day, preferably 1000-4500 kJ per day, most preferably 2000-4000 kJ per day (15-30% reduction relative to normal energy intake). The Daily Balance equation is illustrated in more detail in Example 7 below.

Generally, by using the products of the invention and/or following the inventive dietary regimen weight loss of 0.1 kg to 3 kg, preferably from 0.2 kg to 2 kg, most preferably from 0.5 kg to 1 kg per week is achieved.

The particular virtue of the invention is that even at the overuse of the product, sufficient energy levels are provided, to ensure an adequate energy intake for healthy dieting Furthermore, the right balance between the amount of steroidal glycoside extract and the energy content of the unit serving can be determined using the equation. The inventive products are designed to give good nutrition while providing effective reduction in energy to enable steady weight loss. The Energy Balance Equation is crucial when balancing the opposing aims of providing weight loss and also the required energy intake. By using the Energy Balance Equation, it is possible to design the optimum unit serving products and also to design the regimen of using the unit serving products.

### Optional Ingredients

The inventive composition preferably includes additional nutrients, vitamins and minerals to deliver healthy nutrition, despite the appetite suppression. Suitable vitamins and minerals, include but are not limited to Vitamin A, Vitamin D, Vitamin E, Vitamin C, Thiamin, Riboflavin, Niacin, Vitamin B6, folate, Vitamin B12, Biotin, Pantothenic acid, Calcium, Phosphorous, Potassium, Iron, Zinc, Copper, Iodine, Selenium, Sodium, Magnesium, Manganese, molybdenum, vitamin K, chromium, and mixtures thereof. The preferred ingredients to deliver vitamins and minerals include but are not limited to potassium phosphate, calcium phosphate, magnesium oxide, magnesium phosphate ascorbic acid, sodium ascorbate, vitamin E acetate, niacinamide, ferric orthophosphate, calcium pantothenate, zinc oxide, zinc gluconate, vitamin A palmitate, pyridoxine hydrochloride, riboflavin, thiamin mononitrate, biotin, folic acid, chromium chloride, potassium iodide, sodium molybdate, sodium selenate, phytonadone (vitamin K), cholecalciferol (vitamin D3), cyanocobalamin (vitamin b12), manganese sulfate and mixtures thereof. Preferably, the inventive product contains at least 10% or more of the recommended daily amount (“RDA”) of the vitamins and minerals.

The inventive products may further include meat, fish, meat and fish extracts, fruit, dried fruit, fruit concentrates, fruit extracts, fruit juices, tea (e.g. green tea) vegetables, vegetable extracts and concentrates, nuts, nut extracts, chocolate, bread, vinegar, salt, pepper, cocoa powder, herbs (e.g. parsley), herb extracts, spices (e.g. cinnamon), spice extracts, emulsifiers, acidity regulators (e.g. phosphoric, malic, citric,tartaric acids and salts thereof), flavonoids, preservatives (e.g. lactic acid, EDTA, tocopherols, sodium benzoate), colors (e.g. beta carotene, lycopene, caramel, carmine red), fibers (e.g. soy), leavening agents (e.g., sodium bicarbonate), pectin, citric acid, yeast, salt, glycerine, and mixtures thereof.

The preferred inventive products are substantially free of cholesterol, i.e. the products comprise less than 10 mg of cholesterol, preferably no more than 5 mg per unit serving, and optimally are free of cholesterol. The preferred products include sterols and/or stanols for cholesterol lowering effects.

The preferred inventive products comprise less than 6 g of sodium, preferably less than 3 g, most preferably less than 1 g, optimally less than 150 mg per unit serving.

The preferred inventive products contain at least 70 mg of potassium, preferably at least 100 mg, most preferably at least 140 mg per unit serving.

The product format of the edible appetite suppressant product in unit serving form can be chosen from many formats. For example, it can be a bar weighing from 20 to 100 g, preferably from 40 to 100 g or a drink in a volume of 80 to 500 ml, preferably 90 to 400 ml, most preferably from 100 to 350 ml (taking into account a typical density of drinks of from 0.8 to 1.2 g/ml, the drink would have a weight in grams of from about 60 g to 600 g).

### Process of Preparing

A steroidal glycoside may be incorporated into the inventive unit serving product in the same manner as any food ingredient. Preferably, the steroidal glycoside is finely dispersed, preferably delivered in the extract from plants. The extract is prepared as described above, or by CO₂ extraction, or by any other suitable method.

The most preferred method of preparing a unit serving drink according to the invention comprises the steps of:

(a) preparing a composition comprising at least 1% by weight steroidal glycosides,

(b) dissolving an emulsifier having a HLB (hydrophilic lipophilic balance) value between 7 and 30 in a solvent, and

(c) thoroughly homogenising the composition comprising the steroidal glycosides for about 1 to 10 minutes in the solution of the solvent and the emulsifier at a temperature of 20 to 200° C. to obtain a dispersion, whereby the steroidal glycosides are dispersed to a mean particle size (D3,2) of less than 15 micrometer, wherein the ratio (w/w) of the steroidal glycosides to the emulsifier in the edible dispersion is less than 10:1.

All amounts, parts, ratios and percentages used herein are by weight, unless otherwise specified.

While the above summarizes the present invention, it will become apparent to those skilled in the art that modifications, variations and alterations may be made without deviating from the scope and spirit of the present invention as described and claimed herein. The invention will now be further illustrated in the following non-limiting examples.

### EXAMPLE 1

The following appetite suppressant Muesli Bar, Yoghurt Muesli Variants, is within the scope of the invention:

Formulation:

| | % |
|---|---|
| Ingredient | Formula |
| Maltitol | 4.350 |
| Glucose syrup | 8.403 |
| Polydextrose syrup | 13.344 |
| Inulin syrup | 5.600 |
| Coconut oil | 1.000 |
| Mazola oil | 2.300 |
| Brown Sugar | 1.000 |
| Lecithin | 0.600 |
| Pectose paste | 5.000 |
| Glycerine | 5.000 |
| Date paste | 3.000 |
| Flavourings | 0.405 |
| Colouring | 0.144 |
| Oatflakes | 3.706 |
| Coconut flakes | 1.900 |
| Apple Fiber | 3.900 |
| Soy Nuggets | 31.100 |
| Vitamin & Mineral | 3.914 |
| Premix | |
| Yogurt Coating | 10.000 |
| Plant Extract (80% | 1.000 |
| steroidal glycosides) | |
| Water loss during | −5.666 |
| manufacture | |
| TOTAL | 100.000 |

Nutritional Info:

| | |
|---|---|
| Product weight as consumed: | 60 g |
| Energy density (kJ/g) | 14.62 |

| | Per 100 g | Per 60 g Bar |
|---|---|---|
| Energy | | |
| Energy Value (kJ) | 1465 | 877 |
| Energy Value (kcal) | 347 | 208 |
| % Energy from Protein | — | 27.50 |
| % Energy from Carbohydrates | — | 48.65 |
| % Energy from Fat | — | 28.13 |
| Trans Fat (g) | 0.087 | 0.052 |
| % Energy from Trans Fat | — | 0.23 |
| Linoleic Acid (g) | — | 1.42 |
| Sodium (mg) | 390 | 230 |
| Cholesterol (mg) | N/A | N/A |
| Vitamins | | |
| Vitamin A (μg) | 380.00 | 228.00 |
| Vitamin D (μg) | 3.33 | 2.00 |
| Vitamin E (mg) | 6.70 | 4.02 |
| Vitamin C (mg) | 30.00 | 18.00 |
| Thiamin (mg) | 0.83 | 0.50 |
| Riboflavin (mg) | 0.81 | 0.49 |
| Niacin (mg) | 10.30 | 6.18 |
| Vitamin B6 (mg) | 1.00 | 0.60 |
| Folic Acid (μg) | 140.00 | 84.00 |
| Vitamin B12 (μg) | 1.47 | 0.88 |
| Biotin (mg) | 0.05 | 0.03 |
| Pantothenic Acid (mg) | 2.65 | 1.59 |
| Minerals | | |
| Calcium (mg) | 368.00 | 220.80 |
| Phosphorus (mg) | 534.00 | 320.40 |
| Iron (mg) | 9.60 | 5.76 |
| Magnesium (mg) | 90.00 | 54.00 |
| Zinc (mg) | 5.70 | 3.42 |
| Iodine (μg) | 70.00 | 42.00 |
| Potassium (mg) | 833.34 | 500.00 |
| Copper (mg) | 0.70 | 0.42 |
| Selenium (μg) | 32.50 | 19.50 |
| Manganese (mg) | 0.70 | 0.42 |

### EXAMPLE 2

The following appetite suppressant Chicken & Mushroom Soup is within a scope of the invention:

Formulation:

| | % |
|---|---|
| Ingredient | Formula |
| Water | 67.983 |
| Skimmed Milk Powder | 1.218 |
| Sodium Phosphate | 0.126 |
| Corn Oil | 1.034 |
| Butter Concentrate | 0.528 |
| Wheat Flour | 2.002 |
| Modified Maize Starch | 1.575 |
| Flavourings | 0.567 |
| White Pepper | 0.014 |
| Mace Powder | 0.007 |
| Maltodextrin | 2.800 |
| Titanium Dioxide | 0.182 |
| Garlic Powder | 0.028 |
| Onion Powder | 0.028 |
| Gelatine | 3.150 |
| Salt | 0.399 |
| MSG | 0.280 |
| Vitamin & Mineral | 0.858 |
| Parsley | 0.406 |
| Chicken Meat | 10.544 |
| Mushrooms | 4.461 |
| Cream | 0.811 |
| Plant Extract (80% | 1.000 |
| steroidal glycosides) | |
| TOTAL | 100.00 |

Nutritional Info:

| | |
|---|---|
| Product weight as | 295 ml |
| consumed: | |
| Energy density (kJ/g) | 2.94 |

| | Per 100 ml | Per 295 ml |
|---|---|---|
| Energy | | |
| Energy Value (kJ) | 311 | 918 |
| Energy Value (kcal) | 74 | 218 |
| % Energy from Protein | — | 36.70 |
| % Energy from | — | 34.68 |
| Carbohydrates | | |
| % Energy from Fat | — | 30.14 |
| Trans Fat (g) | N/A | N/A |
| % Energy from Trans Fat | — | N/A |
| Linoleic Acid (g) | — | 1.69 |
| Sodium (mg) | 360 | 1060 |
| Cholesterol (mg) | N/A | N/A |
| Vitamins | | |
| Vitamin A (μg) | 92.54 | 273.00 |
| Vitamin D (μg) | 0.66 | 1.95 |
| Vitamin E (mg) | 1.32 | 3.90 |
| Vitamin C (mg) | 9.15 | 27.00 |
| Thiamin (mg) | 0.14 | 0.40 |
| Riboflavin (mg) | 0.20 | 0.58 |
| Niacin (mg) | 2.20 | 6.50 |
| Vitamin B6 (mg) | 0.18 | 0.54 |
| Folic Acid (μg) | 24.41 | 72.00 |
| Vitamin B12 (μg) | 0.17 | 0.50 |
| Biotin (mg) | 0.002 | 0.005 |
| Pantothenic Acid (mg) | 0.37 | 1.10 |
| Minerals | | |
| Calcium (mg) | 85.42 | 252.00 |
| Phosphorus (mg) | 96.61 | 285.00 |
| Iron (mg) | 1.97 | 5.80 |
| Magnesium (mg) | 18.31 | 54.00 |
| Zinc (mg) | 1.15 | 3.40 |
| Iodine (μg) | 15.93 | 47.00 |
| Potassium (mg) | 186.44 | 550.00 |
| Copper (mg) | 0.14 | 0.40 |
| Selenium (μg) | 6.71 | 19.80 |
| Manganese (mg) | 0.12 | 0.36 |

### EXAMPLE 3

The following appetite suppressant Strawberry Milk Drink is within the scope of the invention:

Formulation:

| | % |
|---|---|
| Ingredient | Formula |
| Skim Milk | 75.300 |
| Water | 12.782 |
| Sucrose | 6.600 |
| Gum Arabic | 1.500 |
| Milk Protein | 1.600 |
| Corn Oil | 0.600 |
| Flavouring | 0.200 |
| Emulsifier | 0.150 |
| Colouring | 0.085 |
| Vitamin & Mineral Premix | 0.182 |
| Plant Extract (80% | 1.000 |
| steroidal glycosides) | |
| TOTAL | 100.00 |

Nutritional Info:

| | |
|---|---|
| Product weight as | 325 ml |
| consumed: | |
| Energy density (kJ/g) | 2.65 |

| | Per 100 ml | Per 325 ml |
|---|---|---|
| Energy | | |
| Energy Value (kJ) | 281 | 914 |
| Energy Value (kcal) | 66 | 216 |
| % Energy from Protein | — | 25.37 |
| % Energy from | — | 63.89 |
| Carbohydrates | | |
| % Energy from Fat | — | 10.83 |
| Trans Fat (g) | 0.006 | 0.02 |
| % Energy from Trans Fat | — | 0.08 |
| Linoleic Acid (g) | 0.32 | 1.03 |
| Sodium (mg) | 60 | 200 |
| Cholesterol (mg) | N/A | N/A |
| Vitamins | | |
| Vitamin A (μg) | 86.00 | 279.50 |
| Vitamin D (μg) | 0.65 | 2.11 |
| Vitamin E (mg) | 1.10 | 3.58 |
| Vitamin C (mg) | 6.50 | 21.13 |
| Thiamin (mg) | 0.23 | 0.75 |
| Riboflavin (mg) | 0.23 | 0.75 |
| Niacin (mg) | 2.70 | 8.78 |
| Vitamin B6 (mg) | 0.28 | 0.91 |
| Folic Acid (μg) | 21.50 | 69.88 |
| Vitamin B12 (μg) | 0.18 | 0.59 |
| Biotin (mg) | 0.02 | 0.05 |
| Pantothenic Acid (mg) | 0.65 | 2.11 |
| Minerals | | |
| Calcium (mg) | 123.00 | 399.75 |
| Phosphorus (mg) | 80.00 | 260.00 |
| Iron (mg) | 1.70 | 5.53 |
| Magnesium (mg) | 20.00 | 65.00 |
| Zinc (mg) | 1.60 | 5.20 |
| Iodine (μg) | 16.20 | 52.65 |
| Potassium (mg) | 154.00 | 500.50 |
| Copper (mg) | 0.15 | 0.49 |
| Selenium (μg) | 7.50 | 24.38 |
| Manganese (mg) | 0.20 | 0.65 |

### EXAMPLE 4

The following appetite suppressant Pomodoro Pasta Meal (for reconstitution) is within the scope of the invention:

Formulation:

| | % |
|---|---|
| Ingredient | Formula |
| Tomato Powder | 12.024 |
| Fat Powder | 5.428 |
| Basil | 0.935 |
| Flavourings | 9.887 |
| Paprika | 0.468 |
| Cheese Powder | 1.069 |
| Sucrose | 1.336 |
| Guar Meal | 0.241 |
| Parsley | 0.267 |
| Pasta | 37.409 |
| MSG | 0.668 |
| Onion Powder | 0.668 |
| Vegetable Protein | 12.720 |
| Potato Starch | 3.275 |
| Whey Powder | 10.020 |
| Thyme | 0.267 |
| Vitamin & Mineral | 1.209 |
| Premix | |
| Garlic Powder | 0.401 |
| Gum Arabic | 0.668 |
| White Pepper | 0.040 |
| Plant Extract (80% | 1.000 |
| steroidal glycosides) | |
| TOTAL | 100.000 |

Nutritional Info:

| | |
|---|---|
| Product weight as consumed: | 71.5 g + 200 ml hot Water |
| Energy density as consumed | 3.66 |
| (kJ/g) | |

| | Per 100 g dry | |
|---|---|---|
| | product | Per serving |
| Energy | | |
| Energy Value (kJ) | 1391 | 995 |
| Energy Value (kcal) | 329 | 235 |
| % Energy from Protein | — | 25.53 |
| % Energy from Carbohydrates | — | 62.64 |
| % Energy from Fat | — | 11.87 |
| Trans Fat (g) | 0.019 | 0.014 |
| % Energy from Trans Fat | — | 0.05 |
| Linoleic Acid (g) | 1.71 | 1.22 |
| Sodium (mg) | 1300 | 900 |
| Cholesterol (mg) | N/A | N/A |
| Vitamins | | |
| Vitamin A (μg) | 419.58 | 300.00 |
| Vitamin D (μg) | 2.14 | 1.53 |
| Vitamin E (mg) | 14.73 | 10.53 |
| Vitamin C (mg) | 83.92 | 60.00 |
| Thiamin (mg) | 1.68 | 1.20 |
| Riboflavin (mg) | 1.06 | 0.76 |
| Niacin (mg) | 11.19 | 8.00 |
| Vitamin B6 (mg) | 0.98 | 0.70 |
| Folic Acid (μg) | 119.86 | 85.70 |
| Vitamin B12 (μg) | 2.38 | 1.70 |
| Biotin (mg) | 0.11 | 0.08 |
| Pantothenic Acid (mg) | 4.41 | 3.15 |
| Minerals | | |
| Calcium (mg) | 335.66 | 240.00 |
| Phosphorus (mg) | 447.55 | 320.00 |
| Iron (mg) | 9.79 | 7.00 |
| Magnesium (mg) | 153.85 | 110.00 |
| Zinc (mg) | 7.44 | 5.32 |
| Iodine (μg) | 54.55 | 39.00 |
| Potassium (mg) | 993.01 | 710.00 |
| Copper (mg) | 1.12 | 0.80 |
| Selenium (μg) | 30.77 | 22.00 |
| Manganese (mg) | 1.54 | 1.10 |

The 80% steroidal glycoside had the following specification;

| Specification item | Specification limit | Test method |
|---|---|---|
| Physical description | Free flowing powder, free from | In-house |
| Form | particulate contamination | |
| Colour | Light Yellow-green | In-house |
| Active components | | |
| Steroidal | >80% | HPLC |
| glycosides | | |
| Water content | <3% | Karl Fischer |
| Residual solvents | | |
| Heptane | <0.5% | |
| Ethanol | <0.5% | |
| Particle size | <50 microns | In-house |
| Microbiological | | Ph. Eur. |
| examination | | |
| Total viable | | |
| aerobic count | | |
| Aerobic | ≦10⁴ cfu/g | |
| bacteria | | |
| Fungi | ≦10² cfu/g | |
| Enterobacteriaceae | ≦10² cfu/g | |
| Escherichia | Absent in 1 g | |
| coli | | |
| Staphylococcus | Absent in 1 g | |
| aureus | | |
| Salmonella spp | Absent in 10 g | |

### EXAMPLE 5

The following appetite reducing acidified dairy drink was prepared;

Formulation:

| | % |
|---|---|
| Ingredient | Formula |
| Skim Milk | 6.750 |
| Water | 79.250 |
| Sucrose | 5.000 |
| HM Pectin | 0.450 |
| Milk Protein | 1.600 |
| Canola oil | 5.000 |
| Citric acid (50% solution) | 1.250 |
| Emulsifier | 0.200 |
| Flavouring | 0.200 |
| Acesulfame-K | 0.050 |
| Aspartame | 0.050 |
| Plant Extract (30% | 0.200 |
| steroidal glycosides) | |
| TOTAL | 100.00 |

Nutritional Info:

| | |
|---|---|
| Product weight as | 90 g |
| consumed: | |
| Energy density (kJ/g) | 3.71 |

| | Per 100 g wet | Per |
|---|---|---|
| | product | serving |
| Energy | | |
| Energy Value (kJ) | 371 | 334 |
| Energy Value (kcal) | 89 | 80 |
| % Energy from Protein | — | 11 |
| % Energy from | — | 38 |
| Carbohydrates | | |
| % Energy from Fat | — | 51 |
| | — | |

The 30% extract had the following specification:

| Specification item | Specification limit | Test method |
|---|---|---|
| Physical description | Powder, free from | In-house |
| Form | particulate contamination | |
| Colour | Yellow-green | In-house |
| Active Components | | |
| Steroidal | >30% | HPLC |
| glycosides | | |
| Water content | <5% | Karl |
| | | Fischer |
| Residual solvents | | |
| Heptane | <1% | |
| Ethanol | <1% | |
| Particle size | <100 microns | In-house |
| Microbiological | | Ph. Eur. |
| examination | | |
| Total viable | | |
| aerobic count | | |
| Aerobic | ≦10⁴ cfu/g | |
| bacteria | | |
| Fungi | ≦10² cfu/g | |
| Enterobacteriaceae | ≦10² cfu/g | |
| Escherichia coli | Absent in 1 g | |
| Staphylococcus | Absent in 1 g | |
| aureus | | |
| Salmonella spp | Absent in 10 g | |

Hoodia gordonii extract water dispersion process: 900 ml demineralised water was heated to 95° C. in a 1000 ml beaker. The water was agitated using a Silverson mixer set at 5,500 rpm. Then, 8 g sodium stearoyl lactylate was added and the mixture was agitated until the stearoyl lactylate was completely dissolved. The shaft speed of the Silverson was then increased to 6,200 rpm and 8 g Hoodia gordonii extract (obtained according to the process of U.S. Pat. No. 6,376,657) containing about 31% (w/w) steroidal glycosides was added and mixed until it was fully dispersed. The dispersion was then cooled below 40° C. by putting the beaker in an ice bath under while continuing mixing at 5000 rpm. Demineralised water was added to a total volume to 1000 ml.

A 4000 ml beaker was filled with 1000 ml of the aqueous Hoodia gordonii dispersion containing 8 g Sodium stearoyl lactylate and 8 g Hoodia gordonii extract and 2200 ml demineralised water. The dispersion was reheated to 75° C. under moderate stirring. The vegetable oil was gently added and mixture was stirred for 2-3 minutes. Then, the skim milk powder, the pectin and the sugars/sweeteners as a powder blend were added and mixed for 5-8 minutes until all lumps had disappeared. During mixing the citric acid solution was slowly added to set the pH of the drink to 4.0. Finally, demineralised water was added to set the total amount to 4000 g.

### EXAMPLE 6

Male Sprague-Dawley rats weighing 210-250 g were obtained from Harlan Italy s.r.l. (San Pietro al Natisone (UD), Italy) and individually housed upon arrival. The animals were maintained under a 12:12 light dark cycle (lights off at 18:00) in a temperature and humidity controlled environment (22±2° C./55±15% humidity). The animals were fed a standard rat chow and had free access to tap water.

After 1 week of acclimatisation, the animals were randomly assigned to groups of 10-15 animals. Each group received by oral gavage either vehicle (0.5% carboxy methyl cellulose (CMC) in water) or Hoodia gordonii extract (30% steroidal glycosides at a dosage of 10, 30 or 60 mg/kg body weight) suspended in vehicle once per day for a minimum of 14 days. Food intake and body weight were measured daily.

Table 1 shows the body weight of rats that received vehicle, 10 mg/kg or Hoodia gordonii extract, 30 mg/kg or Hoodia gordonii extract or 60 mg/kg or Hoodia gordonii extract at day 1 and day 7 of the treatment. Data are presented as means±SD.

TABLE 1

Body weight (g)

| Treatment | Day 1 | Day 7 |
|---|---|---|
| Vehicle | 273 ± 9 | 307 ± 12 |
| 10 mg/kg Hoodia gordonii | 272 ± 8 | 269 ± 15* |
| extract | | |
| 30 mg/kg Hoodia gordonii | 269 ± 9 | 241 ± 9* |
| extract | | |
| 60 mg/kg Hoodia gordonii | 271 ± 9 | 236 ± 11* |
| extract | | |

At day 7 of the treatment, the mean body weights of rats receiving 10, 30 and 60 mg/kg Hoodia gordonii extract was significantly different from rats receiving vehicle (p<0.01, Dunnett's test—denoted by * in Table 1).

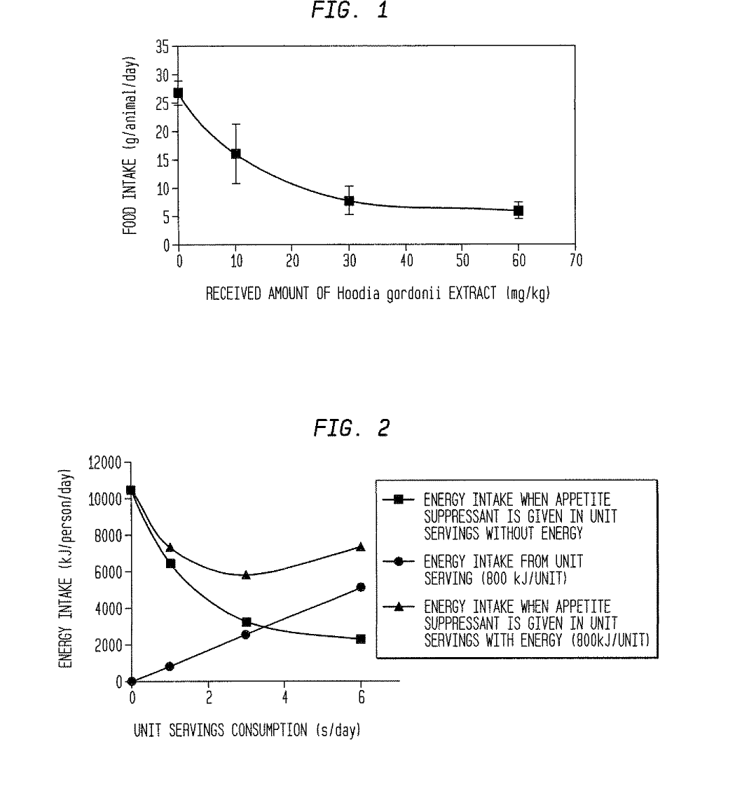

FIG. 1 shows the daily food intake of rats that received vehicle, 10 mg/kg or Hoodia gordonii extract, 30 mg/kg or Hoodia gordonii extract or 60 mg/kg or Hoodia gordonii extract at day 7. Data are presented as means±SD.

### EXAMPLE 7

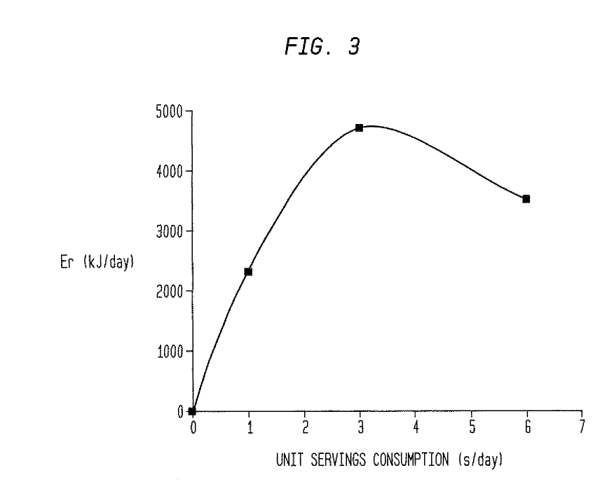

The data from Example 6 were used to model the effects of simultaneous consumption of a nourishing amount of energy and steroidal glycoside on energy intake in humans FIG. 2 shows the decrease in energy intake in humans after consuming increasing numbers of a 0 kJ unit servings containing steroidal glycoside (squares). The data are based on the results in rats as shown in Example 6, assuming a person with an average daily energy consumption of 10,460 kJ. The part of the curve with squares where the curve becomes linear (around 2000 kJ in FIG. 2) represents Eₘ in Energy Balance Equation according to the invention.

When steroidal glycoside is consumed with a certain amount of energy, this energy adds to the total daily energy intake of the person consuming the unit servings. FIG. 2 shows the increase in energy intake from the consumption of increasing numbers of unit servings (curve with circles) containing a certain amount of energy (E, in Energy Balance Equation according to the invention) In this example, Eₛ was set at 800 kJ.

FIG. 2 also illustrates (curve with triangles) the sum of:

(1) the decrease in energy intake resulting from the intake of a certain amount of steroidal glycoside in a unit serving and

(2) the increase of energy intake resulting from the intake of a certain amount of energy in the unit serving.

As shown, the balance between the amount of the steroidal glycoside and the amount of energy in the unit serving determines the maximum decrease in energy intake. The difference between the line with the triangles and the energy intake with 0 unit servings (10460 kJ in FIG. 2) represents Eᵣ in Energy Balance Equation according to the invention.

FIG. 3 shows the curve for Eᵣ, using the data from FIG. 2. As shown in this example, the reduction in energy intake is 2300 to 4700 kJ over a wide range of unit servings consumed per day.

While described in terms of the presently preferred embodiments, it is to be understood that such disclosure is not to be interpreted as limiting. Various modifications and alterations will no doubt occur to one skilled in the art after having read the above disclosure. Accordingly, it is intended that the appended claims be interpreted as covering all such modifications and alterations as fall within the true spirit and scope of the invention.

1-3. (canceled)

4. The method of claim 31, wherein the steroidal glycoside is obtainable from plants of the Asclepiadaceae family.

5. The method of claim 4, wherein the steroidal glycoside is obtainable from genera of plants selected from the group consisting of Hoodia, Caralluma, Orbea, Stapelia, Lavrania, and mixtures thereof

6. The method of claim 4, wherein the steroidal glycoside is obtainable from the plants selected from the group consisting of Trichocaulon piliferum, Trichocaulon officinale, Hoodia currorii, Hoodia gordonii, Hoodia lugardii and mixtures thereof.

7. The method of claim 1 wherein the steroidal glycoside is incorporated into the product in the form of a concentrated preparation obtainable from plants.

8. The method according to claim 7 wherein the concentrated preparation comprises at least about 10% of the steroidal glycoside, by weight of the concentrated preparation.

9. The method of claim 31 wherein the steroidal glycoside is incorporated into the product in the form of an extract obtainable from plants.

10. The method according to claim 9 wherein the extract comprises at least about 10% of the steroidal glycoside, by weight of the extract.

11. The method according to claim 9 wherein the extract comprises at least about 20% of the steroidal glycoside, by weight of the extract.

12. The method according to claim 9 wherein the extract comprises at least about 60% of the steroidal glycoside, by weight of the extract.

13. The method of claim 31 wherein the steroidal glycoside comprises a deoxy or a di-deoxy glycoside.

14. The method of claim 31, wherein the steroidal glycoside has the general Formula (1): 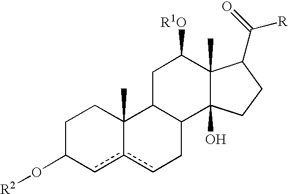
wherein
R=alkyl;
R¹ is selected from the group consisting of H, alkyl, tiglyol, benzoyl and an organic ester group;
R² is selected from the group consisting of H, or one or more 6-deoxy carbohydrates, or one or more 2,6-dideoxy carbohydrates, glucose radical, and mixtures thereof thereof; and wherein the broken lines indicate the optional presence of a further bond between carbon atoms C4 and C5 or between carbon atoms C5 and C6.

15-16. (canceled)

17. The method according to claim 31, wherein fat provides from about 2% to about 45% of the energy density.

18. The method according to claim 31, wherein fat provides from about 5% to about 40% of the energy density.

19. The method of claim 31, wherein from about 10 to about 80%, by weight of the fat, is unsaturated or polyunsaturated oil.

20. The method of claim 31, wherein the product contains at least 1 g of linoleic and/or linolenic acid.

21. (canceled)

22. The method of claim 31, wherein protein provides from about 5% to about 45% of the energy density.

23. The method of claim 31, wherein protein provides from about 10% to about 35% of the energy density.

24-26. (canceled)

27. The method according to claim 31, the method comprising administering from 1 to 5 of the products per day.

28. The method according to claim 31, the method comprising administering from 1 to 3 of the products per day.

29-30. (canceled)

31. A method of achieving reduction in daily energy intake in a human, the method comprising administering orally to the human an edible appetite suppressant product comprising a balanced amount of steroidal glycosides and energy content.
wherein the product is in the form of an individually packaged separate bars, each bar having a weight of from about 20 g to about 100 g and each bar having the energy density from about 10 kJ/g to about 20 kJ/g; and
wherein each bar comprises from about 50 to about 2000 mg of steroidal glycosides; and
wherein each bar comprises a mixture of fats, proteins, and carbohydrates in amounts such as to provide from about 0.5% to about 55% energy from fat, from about 3% to about 50% energy from protein, and from about 10% to about 75% energy from carbohydrate;
whereby the balanced amounts of the steroidal glycoside and the energy density are such as to achieve the reduction in daily energy intake of from about 500 kJ per day to about 5000 kJ per day.

32. A method of achieving reduction in daily energy intake in a human, the method comprising administering orally to the human an edible appetite suppressant product comprising a balanced amount of steroidal glycosides and energy content delivered by a mixture of proteins, fats and carbohydrates,
wherein the product is in the form of an individually packaged separate drinks. each drink having a weight of from about 80 ml to about 500 ml and each drink having the energy density from about 1.6 kJ/g to about 10 kJ/g; and
wherein each drink comprises from about 50 to about 2000 mg of steroidal glycosides; and
wherein each drink comprises a mixture of fats, proteins, and carbohydrates in amounts such as to provide from about 0.5% to about 55% energy from fat, from about 3% to about 50% energy from protein, and from about 10% to about 75% energy from carbohydrate;
whereby the balanced amounts of the steroidal glycoside and the energy density are such as to acheive the reduction in daily energy intake of from about 500 kJ per day to about 5000 kJ per day.

33. The method according to claim 32, wherein the steroidal glycoside is obtainable from plants of the Asclepiadaceae family.

34. The method according to claim 32, wherein the steroidal glycoside is obtainable from genera of plants selected from the group consisting of Hoodia, Caralluma, Orbea, Stapelia, Lavrania, and mixtures thereof.

35. The method according to claim 32, wherein the steroidal glycoside is obtainable from the plants selected from the group consisting of Trichocaulon piliferum, Trichocaulon officinale, Hoodia currorii, Hoodia lugardii and mixtures thereof.

36. The method according to claim 32 wherein the steroidal glycoside is incorporated into the product in the form of a concentrated preparation obtainable from plants.

37. The method according to claim 32 wherein the concentrated preparation comprises at least about 10% of the steroidal glycoside, by weight of the concentrated preparation.

38. The method according to claim 32 wherein the steroidal glycoside is incorporated into the product in the form of an extract obtainable from plants.

39. The method according to claim 32 wherein the extract comprises at least about 10% of the steroidal glycoside, by weight of the extract.

40. The method according to claim 32 wherein the extract comprises at least about 20% of the steroidal glycoside, by weight of the extract.

41. The method according to claim 32 wherein the extract comprises at least about 60% of the steroidal glycoside, by weight of the extract.

42. The method according to claim 32 wherein the steroidal glycoside comprises a deoxy or a di-deoxy glycoside.

43. The method according to claim 32, wherein the steroidal glycoside has the general Formula (1): 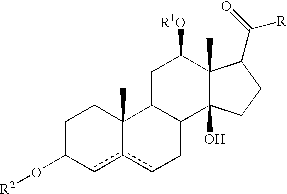
wherein
R=alkyl;
R₁ is selected from the group consisting of H, alkyl, tiglyol, benzoyl and an organic ester group;
R₂ is selected from the group consisting of H, or one or more 6-deoxy carbohydrates, or one or more 2,6-dideoxy carbohydrates, glucose radical, and mixtures thereof thereof; and wherein the broken lines indicate the optional presence of a further bond between carbon atoms C4 and C5 or between carbon atoms C5 and C6.

44. The method according to claim 32, wherein fat provides from about 2% to about 45% of the energy density.

45. The method according to claim 32, wherein fat provides from about 5% to about 40% of the energy density.

46. The method according to claim 32, wherein from about about 10 to about 80%, by weight of the fat, is unsaturated or polyunsaturated oil.

47. The method according to claim 32, wherein the product contains at least 1 g of linoleic and/or linolenic acid.

48. the method according to claim 32, wherein protein provides from about 5% to about 45% of the energy density.

49. The method according to claim 32, wherein protein provides from about 10% to about 35% of the energy density.

50. The method according to claim 32, the method comprising administering from 1 to 5 of the products per day.

51. The method according to claim 32, the method comprising administering from 1 to 3 of the products per day.
